(19) United States
(12) Patent Application Publication
(10) Pub. No.: US 2004/0185063 A1
(43) Pub. Date: Sep. 23, 2004

(54) Broken cell wall chlorella and process for preparation thereof

(76) Inventor: Timothy Nicholas Ray, Los Angeles, CA (US)

Correspondence Address: Thomas P. Liniak, Liniak, Berenato & White, LLC, Suite 240, 6550 Rock Spring Drive, Bethesda, MD, 20817, US

(21) Appl. No.: 10/802,766

(22) Filed: Mar. 18, 2004

Related U.S. Application Data
(60) Provisional application No. 60/455,418, filed on Mar. 18, 2003.

(51) Int. Cl. A61K 35/80

(52) U.S. Cl. 424/195.17

(57) ABSTRACT

The present invention is directed to a solution comprising water, alcohol and homogenously dispersed Chlorella diatoms having a diameter of less than about 5 microns. The present invention also relates to a method of pulverizing Chlorella. A solution comprising alcohol and chlorella is maintained at a temperature of less than about zero degrees Celsius while the solution is sonicated for a sufficient period to achieve a solution with homogenously dispersed chlorella diatoms having a diameter of less than about 5 microns.

### CROSS-REFERENCE TO RELATED APPLICATION AND CLAIM TO PRIORITY:

This application is based on provisional application Serial No. 60/455,418, filed Mar. 18, 2004, the disclosure of which is incorporated herein by reference and to which priority is claimed under 35 U.S.C. §120.

### FIELD OF THE INVENTION

The present invention is directed to a process for breaking of the cell walls of Chlorella and the resultant broken wall Chlorella.

### BACKGROUND OF THE INVENTION

Chlorella belongs to the eucaryotic cell category of algae and lives in fresh and marine water as a single celled plant. Its size is about that of a human erythrocyte (i.e. between 2-8 microns in diameter). The name chlorella derives from two Latin words meaning ‘leaf’ (green) and ‘small’, referring to the unusually high content of chlorophyll which gives chlorella its characteristic deep emerald-green color.

Chlorella has the highest content of chlorophyll of any known plant. It also contains vitamins, minerals, dietary fiber, nucleic acids, amino acids, enzymes, etc. It contains more than 9% fats (out of which polyunsaturated fatty acids [PUFA] represent about 82%). The vitamin content consists of provitamin A, vitamins B₁, B₂, B₆, niacin, B₁₂, biotin, vitamin C, vitamin K, pantothenic acid, folic acid, choline, lipoic acid, ionositol, PABA and the like. Among the minerals present, are P, K, Mg, S, Fe, Ca, Mn, Cu, Zn and Co. The main components of Chlorella cells are about 60% protein (composed of all basic amino acids), and 20% carbohydrate. Ingredients related to heavy metal and chemical chelation are the carotinoids and sporopollenin.

Thus, Chlorella is rich in protein, vitamins, minerals, and other beneficial substances and as such has been used successfully for decades as a human nutrient. However, due to its tough cell wall, it is difficult to digest, and often times a significantly greater amount of chlorella must be ingested in order to provide the desired beneficial effect.

Chlorella, in its natural state, has a very tough cell wall. Many efforts have been made to crack or break the cell wall of chlorella to achieve a higher digestibility without adversely impacting or denaturing the chlorella components.

Mechanical means for doing so have included using a grinding or milling process to grind up the chlorella to break the cell wall. During the milling process, the chlorella is exposed to oxygen and higher heat, which may damage the nutrients.

Chemical means for breaking the cell wall have included using enzymes to lyse the cell walls. However, this method can result in disintegration or dissolving of the cell wall and/or denaturing of desired cell wall components.

Other efforts have included developing a genetic strain of chlorella that has a naturally softer cell wall to render the chlorella more digestible.

All of these prior art efforts have required elaborate processes, dangerous conditions, are expensive and time consuming, adversely impact the desired cell wall components, and are highly inefficient due to the extremely tough nature of the chlorella cell walls.

In addition to its high nutrient effects, chlorella also had the capability of attracting and adhering heavy or precious metals to its cell walls. The mining industry has used chlorella to reclaim metals. Chlorella is mixed with water and pumped into a mine shaft or area where it contacts heavy or precious metals that then adhere to its cell wall, and that the heavy or precious metal coated chlorella is pumped out and the metals reclaimed.

However, efforts to adapt the use of chlorella for the purpose of binding to and causing the elimination of heavy metals in the human body has heretofore met with failure.

Taking the prior art ‘cell wall broken’ chlorella does not show a significant increase in the fecal or urinary elimination of heavy metals as compared to baseline, per David Quigg PhD, Technical Director at Doctor's Data Laboratories. Many other doctors, including myself, have found the same thing: normal, over the counter ‘cell wall broken’ chlorella as it is sold today is a difficult to digest broad spectrum nutrient with limited to no ability to chelate heavy metals in the human body.

The question remains: Why does it work so well then in mining? The answer, as you will see, is the ratio of available surface area of the chlorella cell wall to the amount of metals to be absorbed, or chelated. In order to achieve the same effect with a human being as in mining, where hundreds of gallons of chlorella ‘sludge’ are pumped into and basically fill and saturate a mine shaft, a person would have to consume a proportional amount of chlorella, similar to 60% of their body weight, which is impossible to do.

The most desirable form of chlorella would be a chlorella that is ‘cell wall broken’ so that the body is assisted in digesting it and thus absorbing its nutrients. However, the Chlorella cell wall is extremely resistant to destruction and fragmentation. Conventional methods of pulverizing chlorella have not resulted in a truly decimated Chlorella, wherein Chlorella clusters are broken apart, and individual Chlorella diatoms are nanonized into fragments.

Some conventional processes have resulted in broken clusters, wherein the resulting Chlorella clusters have a diameter of about 30 microns or more. For example, attempts to use ultrasonic dismembration have resulted in smaller clusters compared to the initial cluster size of the Chlorella. To achieve even partial breakage of clusters typically takes dozens of hours of processing, filtration, reprocessing, and pre-grinding. However, a single Chlorella diatom has a diameter of about 3-4 microns. Thus, Chlorella having such clusters, often labeled commercially as ‘broken cell wall’ Chlorella, is in fact not truly ‘cell wall broken’. Furthermore, many prior attempts to produce ‘cell wall broken’ Chlorella have resulted in denatured Chlorella lacking the desired nutritional qualities.

### SUMMARY OF THE INVENTION:

The present invention is directed to a solution comprising water, alcohol and homogenously dispersed Chlorella diatoms having a diameter of less than about 5 microns.

The present invention is also directed to nanonized cell wall chlorella powder comprising cell wall fragments having a diameter of less than about 2 microns.

A method of pulverizing Chlorella is disclosed. A liquid solution comprising alcohol and chlorella is provided. The solution is maintained at a temperature of less than about zero degrees Celsius while sonicating the solution using an ultrasonic dismembrator for a sufficient period to achieve a solution with homogenously dispersed chlorella diatoms having a diameter of less than about 5 microns. The disclosed method may also include a further step of fermenting the solution following sonication.

The present invention also relates to a method of removing heavy metals from a patient. A liquid solution is orally administered to the patient. The solution comprises chlorella fragments having a diameter of less than about 5 microns. The fragments bind to heavy metals, such as mercury, in the patient and are thereafter excreted via the patient's urinary tract.

### BRIEF DESCRIPTION OF THE FIGURES:

FIG. 1 is a micrograph showing Chlorella clusters having a diameter of 30 microns or greater;

FIG. 2 is a micrograph showing Chlorella processed according to one embodiment of the present invention;

FIG. 3 is a micrograph showing Chlorella processed according to another embodiment of the present invention.

### DETAILED DESCRIPTION OF THE INVENTION

The present invention is directed to a method of increasing the surface area of a nutriceutical, such as Chlorella clusters, by maintaining the nutriceutical at a temperature of less than about zero degrees Celsius while it is sonicated using ultrasonic dismembration. The nutriceutical may also be fermented prior to the dismembration stage by probiotic predigestion. The resulting cell walls of the chlorella are pulverized and decimated into tiny fragments that are observable with a microscope to be in the range of about 2 microns to less than about 0.1 microns. This decimation substantially increases nutrient availability, given intact chlorella clusters are relatively difficult for humans to fully digest.

Furthermore, the process increases the surface area of the cell wall and thus dramatically increases its ability to bind to a host of heavy metals and other toxins via its ion exchange resin activity. The ingredients in the cell wall that provide this effect are identified as carotenoids in the cell wall and sporopollenin from the interior of the cell, which become much more bioavailable once the cell wall is ‘pulverized’ and the full content of sporopollenin is released into solution.

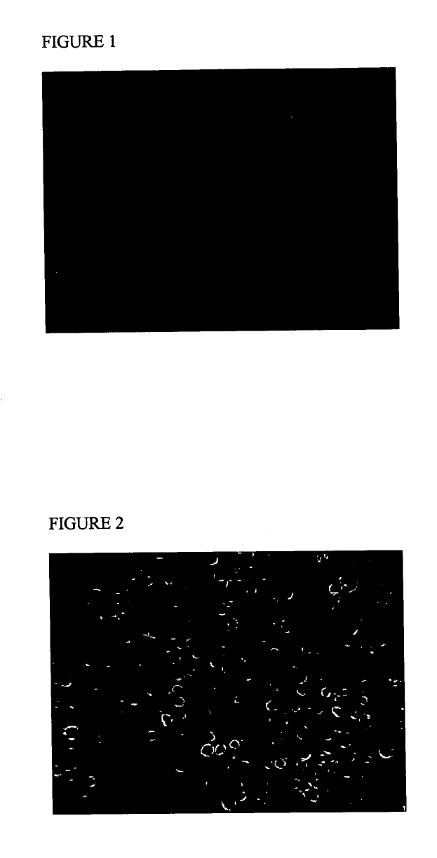

FIGS. 1-3 are micrographs of Chlorella viewed through a DarkField microscope at 400× power, showing Chlorella at various stages of the disclosed method. As best shown in FIG. 1, commercially available Chlorella clusters, advertised as being ‘cell wall broken’, were mixed with water and viewed under the microscope. The micrograph clearly shows that not only are the cell walls intact, but the individual diatoms are tightly clustered in groups of about 500 units each. Each cluster size has a diameter of about 30 microns. Such clusters would likely be very difficult to digest. Indeed, some people get gastrointestinal distress when taking normal, commercially available Chlorella.

Thus, what is termed ‘cell wall broken’ by the companies that manufacture and sell chlorella, as observed with a microscope, possibly means slightly ‘cracked’ or ‘fractured’, but certainly not truly fragmented or even broken into individual diatoms. Conventional methods of ‘breaking’ chlorella result in clusters of the chlorella diatoms having diameters in excess of 30 microns, with a minimal percentage of individual Chlorella diatoms being free in a solution. Possibly what such companies mean by ‘cell wall broken’ is ‘smaller clumps’ of diatoms. In other words, it is not ‘cell wall broken’ at all, and if it is even cracked of fractured a little bit, this is not visible with a light microscope, which would normally be visible as the individual diatoms are clearly observable at 3-4 microns in diameter (slightly smaller than a human red blood cell, which is clearly observable through a microscope).

As best shown in FIG. 2, the Chlorella clusters have been broken apart into separate chlorella diatoms after partial processing according to the disclosed method. Initial decimation of the cell walls is also shown in this micrograph. Some colloids of the cell walls can be seen in the surrounding liquid, the nanocolloids remaining invisible to the light microscope at this stage.

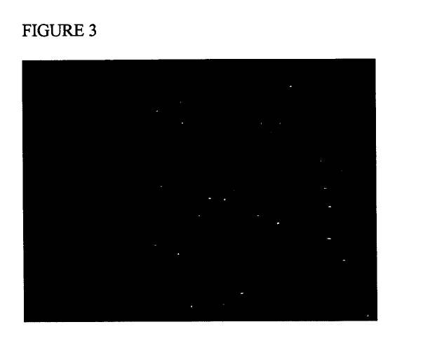

As best shown in FIG. 3, the Chlorella diatoms are pulverized into fragments after final processing according to the disclosed method. The micrograph of FIG. 3 shows the resulting solution after centrifugation, and reveals the resulting colloid fragments of the chlorella. The decimation of the single diatoms is a process referred to herein as nanonization, which simply refers to reducing the size of the particles to the nanometer range whereby the resulting fragments are substantially smaller. Note that there are no single diatoms or clusters shown in FIG. 3. By contrast, there are no nanonized fragments shown in the micrograph shown in FIG. 1. It should be noted that the image shown in FIG. 3 of Chlorella after nanonization does not capture the exact detail of what may be seen through the microscope: millions of scintillating particles having a diameter of about 0.1 microns, surrounded by a haze of even smaller particles.

In addition to data obtained using a light microscope, commercially available broken cell wall Chlorella and nanonized Chlorella processed according to the disclosed method were tested for their abilities to bind to a heavy metal. As the clusters are broken into separate diatoms, and the diatoms are pulverized into fragments, the cell wall surface area increases its ability to bind to a host of heavy metals and other toxins via its ion exchange resin activity. As noted above, the ability to bind to a heavy metal increases as the available surface area of cell wall material increases.

Specifically, samples of commercially available Chlorella (as shown in FIG. 1) and nanonized Chlorella (as shown in FIG. 3) were tested for their ability to bind to methyl mercury vapor using an atomic absorption spectrometer. An absorption spectrometer suitable for such testing is available from Genesis Laboratory Systems, Inc. The absorption spectrometer indicates that Chlorella processed according to the disclosed method, with cell wall fragments having a diameter in the range of between about 2 microns and less than about 0.1 microns, absorbs 50 times more methyl mercury compared to an equivalent amount of commercial cell wall broken chlorella. Both samples were tested under the same conditions of temperature, moisture and pH.

Specific test data is outlined in my published article “The Mitigation of Methyl Mercury Vapor Inhalation and Exhalation in People with Dental Amalgam Fillings”, Townsend Letter for Doctors and Patients, November 2002, the disclosure of which is incorporated herein by reference. As set forth therein, the use of nanonized Chlorella may be used to detoxify heavy metals that leach from amalgam dental fillings in a person, thereby minimizing the release of methyl mercury into the person's gums, sublingual capillary blood flow, and/or digestive tract when mixed with saliva, food or beverages.

Amalgams contain 50% mercury, 35% silver, 9% tin, 6% copper and a trace of zinc. A single dental amalgam filling with a surface area of only 0.4 sq. cm is estimated to release as much as 15 micrograms of mercury per day primarily through mechanical wear and evaporation, The average individual has eight amalgam fillings and could absorb up to 120 micrograms (0.120 mg/m3) of mercury per day from their amalgams.

The primary route of mercury absorption into the body is through the inhalation of mercury vapor. The mercury vapor from the amalgams is lipid soluble and passes readily through cell membranes and across the blood brain barrier. The human body retains approximately 75% of the mercury that is inhaled. Animal studies show that radioactively labeled mercury released from ideally placed amalgam fillings appears quickly in the kidneys, brain and wall of the intestines. The mercury escapes continuously during the entire life of the filling primarily in the form of vapor, but also abraded particles. Chewing, brushing, and the intake of hot fluids stimulate this release. Gold placed in the vicinity of an amalgam restoration produces a 10-fold increase in the release of mercury.

The current ADA estimate that only 0.08 micrograms of mercury per amalgam per day is taken into the human body does not take into consideration that up to five-sixths of the mercury released would be into the tooth (that area of the amalgam that exists below the visibly exposed amalgam surface) and not into the oral air. In addition, some mercury in the oral air would be rapidly absorbed into the saliva and oral mucosa (mercury loves hydrophobic cell membranes) and also not be measured by the mercury analyzer. The ADA estimate does not include the increase that would occur with amalgams in the mouth when chewing, grinding the teeth, drinking hot liquids or in the presence of galvanism, which all greatly increase the release of mercury. Further, as the mercury analyzer pulls mercury containing oral air into the analysis chamber, mercury free ambient air rushes into the oral cavity decreasing the mercury concentration. Taking all of this into account you can calculate that most mercury analyzers could not detect this “estimated” 0.08 micrograms/day level of mercury even if you had several amalgams. However, the fact is that it is quite easy to detect mercury emitting from one amalgam using these (mercury vapor) analyzers. Therefore, the “estimate” by this ADA spokesman is way too low.”

Mercury is neurotoxic to some degree at any level, and has pernicious synergistic effects in combination with many forms of bacteria, other metals, and chemicals. Though we can measure exposure and excretion levels, we cannot yet measure cumulative body burden levels. Mercury has a half-life of between 15-30 years.

Experimental Data:

The oral cavities of persons having amalgam dental fillings were measured with the Hg253 at rest, after chewing gum, and after brushing and rinsing with various substances. Peak values are reported in milligrams per meter cubed (mg/m3). Measurements were taken through a tube placed in the center of the oral cavity while the lips were closed and the person breathed through the nose. The number and age of amalgams, and the presence or absence of gold fillings were recorded as a reference.

A solution having processed Chlorella (referred to in the table as “NDF”) according to the present invention (a 10 drop dose containing 10 mgs. nanonized chlorella) was compared to 10 mg of normal ‘cell wall broken’ chlorella and then again to 100 and 500 mg of normal chlorella, all mixed with water. Reverse osmosis water was used for the control.

TABLE 1

| | #/age of | Gold fillings | Resting | Post chewing | Post wash | Amount | |
|---|---|---|---|---|---|---|---|
| Substances | amalgams | y/n | mg/m3 | mg/m3 | mg/m3 | used | % |
| NDF | 6/30 | 2 | <.001 | .016 | <.001 | 10 dr. | 100% |
| Chlorella (test 1) | 3/15 | N | <.001 | .014 | .010 | 10 mg | 28.6% |
| Chlorella (test 2) | 3/15 | N | <.001 | .014 | .007 | 100 mg | 50% |
| Chlorella (test 3) | 10/16 | N | .054 | .077 | .041 | 500 mg | 100% |
| MouthMagic | 3/15 | N | <.001 | .018 | .005 | 1 oz | 72% |
| Vitamin C | 5/10 | N | <.001 | .011 | .005 | 300 mg | 45% |
| Control | | | | | | | |
| R/O water rinse only | 3/15 | N | <.001 | .013 | .011 | 1 oz | 15% |
| R/O water Brush, Rinse | 10/15 | N | .002 | .116 | .054 | 1 oz | 53% |
| & Spit | | | | | | | |

Normal chlorella finally performed equally to NDF, but at a 500 mg dose, which required brushing and rinsing with the entire quantity, and then rinsing again with r/o water. This was extremely messy and distasteful to the patient as compared to a mere 10 drops of NDF. Interesting to note that it took 50 times more normal chlorella to bind as much mercury vapor as NDF. This explains why most of the testing done with normal chlorella has shown it to be ‘lacking’ as a heavy metal chelator.

Following the selection of the most convenient and efficient method based on the above study, the following study was conducted with 19 people. 5 showed no detectable elevation of mercury after chewing (possibly harder, older fillings as all of these people had had them in for between 20-50 years). 2 did not have time to brush, rinse and re-test. The remaining 12 are reported below. The oral cavities of all persons in the study group were measured with the Hg253 at rest, after chewing, and after brushing and rinsing with either 5 or 10 drops of NDF.

TABLE 2

| | | #/age of amalgams | Gold fillings | Resting S | Resting | Post chewing | Post wash | |
|---|---|---|---|---|---|---|---|---|
| Code | Age | in years | y/n | pH | mg/m3 | mg/m3 | mg/m3 | % |
| Dose: 10 | | | | | | | | |
| drops NDF | | | | | | | | |
| SR | 35 | 6/22 | N | 6.3 | <.001 | 0.05 | <.001 | 100% |
| KR | 50 | 6/37 | N | 6.5 | .002 | .005 | .002 | 100% |
| DD | 32 | 10/16 | N | 7.4 | .083 | .131 | .040 | 100% |
| GL | 64 | 6/30 | 2 | 7.1 | <.001 | .016 | <.001 | 100% |
| JT | 47 | 5/32 | N | 6.4 | <.001 | .019 | <.001 | 100% |
| LW | 28 | 12/12 | N | 6.4 | <.001 | .023 | <.001 | 100% |
| AS | 38 | 11/30 | N | 7.3 | <.001 | .019 | <.001 | 100% |
| LM | 42 | 2/30 | 1 | 6.0 | <.001 | .004 | <.001 | 100% |
| Dose: 5 drops | | | | | | | | |
| NDF. | | | | | | | | |
| DD | 32 | 10/16 | N | | .097 | .131 | .073 | 100% |
| LB | 32 | 7/8 | N | | <.001 | .51 | .17 | 66%* |
| EB | 22 | 5/10 | N | | <.001 | .016 | .002 | 87%** |

*brushed for 30 seconds, also notice the massive release of mercury in this person with newer fillings.
**brushed for 3 minutes

The American Conference of Governmental Industrial Hygienists (ACGIH) has established a threshold level value of 0.025 mg/m3 of mercury for an eight hour time period. The ACGIH additionally recommends that women of childbearing age should not be exposed to air concentrations of mercury greater than 0.010 mg/m3. Additional regulatory agency guidelines for mercury exposure levels are as follows. The Mine Safety and Health Administration (MSHA), National Institute for Occupational Safety and Health (NIOSH), and the World Health Organization (WHO) have established an exposure limit of 0.050 mg/m3 for an eight-hour time period. The Occupational Safety and Health Administration (OSHA) has established a ceiling (peak) exposure level of 0.100 mg/m3.

TABLE 3

| Organization | Threshold | Conversion | Time Frame | Notes |
|---|---|---|---|---|
| ACGIH | 0.025 mg/m3 | 3 ppb | 8 hours | |
| MSHA, | 0.050 mg/m3 | 6 ppb | 8 hours | |
| NIOSH, | | | | |
| WHO | | | | |
| OSHA | 0.100 mg/m3 | 12.1 ppb | peak | Ceiling |
| | | | | exposure |
| | | | | limit |
| ACGIH | 0.010 mg/m3 | 1.21 ppb | peak | Women of |
| | | | | childbearing |
| | | | | age |

NDF can be used to safely and effectively rid the oral cavity of precipitated mercury vapor after chewing, eating, brushing or otherwise disturbing teeth containing amalgam fillings. The dose required during brushing can be estimated from the above data according to number and age of amalgams, relative hardness of amalgams, normal length of chewing, and duration of brushing. In general, the less they use (5 drops), the longer they have to brush, rinse and spit (3-4 minutes). The more they use (10 drops), the shorter the cleaning time (30 seconds to 1 minute).

During detoxification, while amalgams are still in the teeth, the patient brushes with 10 drops NDF, spits and then rinses with r/o water before taking the dose of NDF as drops down the back of the throat, followed by a glass of pure water. Because up to ⅚ of the surface of an amalgam can be inside the tooth, and thus not out gassing into the oral cavity, this procedure is therefore not a complete alternative to having amalgam fillings replaced. It does however minimize and mitigate the inhalation exposure.

Urine and fecal elimination studies were also performed on patients before taking decimated Chlorella, and after taking decimated Chlorella. The results confirmed an increase in heavy metal elimination via the urine, and a decrease via the bowel. This was a vast improvement over the previously recorded effect of over the counter Chlorella.

According to a first embodiment of the disclosed method, a sample of commercially available Chlorella clusters is mixed with alcohol to form a solution. Preferably, the solution includes about 80% by weight of alcohol. The solution is cooled to less than 0° C. but maintained in a liquid state. The liquid solution is then sonicated using an ultrasonic dismembrator while maintaining the temperature below 0° C.

The solution may be placed into a recirculating system, peristaltic pump driven, that passes by the horn of a 500-watt ultrasonic dismembrator in a closed, sterile chamber. The solution is super cooled with 80% alcohol at −15° C., with a second recirculating, pump driven system to offset the heat generated by the ultrasonic dismembrator. Both units are placed into a custom built freezer, which maintains the temperature at −15° C.

The temperature of the chlorella solution is maintained below freezing, preferably at about −1° C. The subzero temperature makes the cell walls brittle and thus more susceptible to breakage with the ultrasonic dismembrator. It is much easier to crack something that is frozen and brittle and inflexible than something that is warm and flexible. This is a natural phenomenon. The temperature of the freezer interior is maintained at −15° C. The freezer temperature, along with the 80% alcohol cooling solution, offset the heat generated by the dismembrator and keep the chlorella at below freezing.

The alcohol content allows the liquid solution to be maintained at a temperature below 0° C. without freezing. Further, the relatively low temperature at which the solution is dismembrated preserves the enzymes that would otherwise be destroyed by the heat generated by conventional ultrasonic processes. Further, the disruption of the clusters and diatom cell walls is enhanced because the cell walls become brittle at subzero temperatures during dismembration. In addition, the resulting chlorella diatoms maintain their greenish color, as opposed to the brownish color prevalent in ‘broken cell wall’ chlorella produced by conventional techniques, due to the subzero temperature processing which maintains its enzymatic and nutritional characteristics. Chlorella solution produced according to the disclosed method undergoes less oxidation, and has a better shelf life.

The solution undergoes ultrasonic dismembration for a period of time sufficient to un-clump the Chlorella clusters until substantially all of the Chlorella are in single diatom form homogenously dispersed throughout the solution, as best shown in FIG. 2. Each diatom has a diameter of between about 3 microns and about 5 microns. Ultrasonic dismembration is stopped before the diatoms begin to fragment, or prior to nanonization.

Typically, the solution should be sonicated using ultrasonic dismembration for about 6 hours or less. Further sonication may fragment the single diatoms, resulting in fragments with diameters of less than 2 microns. Particles smaller than 2 microns may be absorbed directly through the blood gut barrier. However, single diatoms having a diameter of 3 microns or greater will not be absorbed through the blood gut barrier. Rather, such diatoms target detoxification (i.e. binding of heavy metals) of only the gastrointestinal tract and are ultimately excreted through the GI tract.

According to a second embodiment, the alcohol-Chlorella solution is sonicated for a period sufficient to nanonize between about 40% by weight to about 60% by weight of the Chlorella in the solution. Therefore, between about 40-60% of the Chlorella will by decimated into particles having diameters between about 2 microns and about less than 0.1 microns. The remainder of the Chlorella will be in single diatom form with diameters between about 3 microns and about 5 microns. In order to achieve this level of decimation, the solution is typically sonicated for a period of about 18-20 hours. As in the first embodiment, the solution is maintained in a liquid state at a temperature less than 0° C., preferably about −1° C., during ultrasonic dismembration.

Preferably, probiotics are added to the alcohol-Chlorella solution prior to sonication. A proprietary culture of exclusively human beneficial intestinal flora, such as PolyFlor™ or PolyGest™, are suitable probiotics. The resulting solution is then sonicated as described above. Cell wall fragmentation occurs in both the Chlorella, as well as the probiotics. Preferably, the Chlorella cell walls and probiotic cell walls are decimated into particles having diameters of less than 2 microns, or even particles having diameters less than 0.1 microns.

According to a third embodiment, an alcohol-Chlorella solution is sonicated using an ultrasonic dismembrator while maintaining the solution in a liquid state at a temperature less than 0° C., preferably about −1° C., as in the first two embodiments. The solution is sonicated for a period of about 18-20 hours. The resulting dismembrated solution is then fermented via probiotic predigestion. A first portion of probiotics may also be added to the solution prior to sonication, as in the second embodiment. A second portion of probiotics may be added after sonication, wherein the solution then undergoes predigestion.

Decimation of the Chlorella cell wall prior to predigestion permits fermentation. A proprietary culture of exclusively human beneficial intestinal flora, such as PolyFlor™ or PolyGest™, may be used for fermentation stage. Yeast (saccromyces cerveciae) may also be used, but is not required. The fermentation stage typically takes about 18 hours because the cell walls are partially decimated during the dismembration stage. By comparison, conventional methods of fermenting Chlorella typically takes about 72 hours, given the Chlorella diatoms are clustered as noted above. Furthermore, conventional fermentation techniques fail to achieve Chlorella diatoms or fragments, as noted above. Thus, time and costs are substantially reduced, and a truly broken cell wall Chlorella is achieved. Fermentation is considered complete after the pH of the solution reaches about 3.5.

The decimated Chlorella cell wall material may also be softened in an acidic bath prior to fermentation for enhanced probiotic predigestion.

The fermentation stage further nanonizes the cell wall material in the solution, thereby increases its ability to bind to heavy metals. For example, fermented Chlorella solution produced according to the method of the third embodiment increases its ability to bind methyl mercury (as measured with an atomic absorption spectroscopy) by a factor of two compared to an unfermented, decimated chlorella solution produced according to the method of the second embodiment. As such, the chlorella solution produced according to the third embodiment (i.e. sonicated and fermented) is 100 times more effective at binding heavy metals compared to commercially available chlorella clusters.

In any of the methods disclosed herein, the resulting solution may also be subjected to a third grinding stage. I found that high speed rotary grinding of chlorella and probiotics produces a certain low percentage but clinically useful amount of the cell wall broken constituents of each. This process did not actually further break the cell wall of chlorella, but released the already broken cell wall components of diatoms into solution. Following grinding, the solution may be filtered through a 3 micron filter, and the filtrate reclaimed.

The solution may include additional components, added either prior to dismembration or after dismembration and/or fermentation. For example, the solution may include mycelials, herbs, and sea salt.

An exemplary formulation of the solution prior to sonication and fermentation is as follows: A total solution of 8 gallons (˜30.28 liters) includes about 20% alcohol, 820 grams Chlorella, 190 grams probiotics, and 32 ounces (˜907.2 grams) Cilantro.

Following one of the above-disclosed methods, the resulting solution is mixed with a second solution containing alcohol, preferably grain neutral spirits in the amount of about 40% by weight of the second solution, water preferably in the amount of about 50% by weight of the second solution, and tincture of Cilantro preferably in the amount of about 10% by weight. The alcohol serves to lower the surface tension and provides for ease of assimilation, and also acts as a preservative.

In addition, the final mixture used for a particular patient may be a mixture of solutions derived from one or a combination of the embodiments described above. For example, a mixture may include 50% by weight of solution produced from the method according to the second embodiment, and 50% by weight of solution produced form the method according to the third embodiment. Each of the embodiments described above will achieve a different level of cell wall nanonization. As such, the level at which the resultant solution will chelate heavy metals may be controlled depending on percentage by weight of nanonized Chlorella. Furthermore, the target area of detoxification may also be controlled depending on the level of nanonized Chlorella in the resultant solution. For example, a mixture comprising primarily single diatom Chlorella produced via the method of the first embodiment, may be used to target the GI tract for detoxification.

The decimation of Chlorella cell wall via ultrasonic dismembration at subzero temperature along with probiotic predigestion yield a better clinical effect with less waste of chlorella and an 80% yield on a one-pass basis. Yield could be further improved with additional passes through the process of the present invention. By contrast, prior art techniques such as separation and filtering off of pre-existing cell wall fragments provides a 5% yield, with 95% waste.

According to a fourth embodiment, the Chlorella is first immersed in an acidic bath having a pH of about 4.0, and ultra pure reverse osmosis water at a viscous consistency. The acidity softens and opens the cell wall. 208 grams of chlorella, 48 grams of Polyflor (mixed probiotics) are placed into 1 liter of reverse osmosis water. Polyflor, a combination of strains of 26 beneficial human intestinal flora including acidophilus, bifidus, strep thermophilus, are added and mixed into the solution. They are able to feed on the now weakened chlorella and a fermetation process is begun and allowed until the pH of the solution reaches pH 3.5.

The Polyflor component is added to the product for 2 reasons: 1) At the first stage it is used to probiotic predigest the prepared chlorella. 2) Following that it is also put through the dismembration process which kills it for two reasons: breaking its cell wall releases the bacteriocin content and thereby gives the recipient the benefit of the ‘competitive exclusion effect’ of bacteriocins on pathogenic micro organisms such as candida albicans in the GI tract. Killing the probiotics is also a safety feature as they have been cited in the scientific literature as being capable of methylating mercury, not something a person with leaking mercury-amalgam fillings or heavy metal toxicity should risk doing.

The mixture is homogenized in a food quality blender with sharp cutting blades at a speed of 34,000 rpms until the large clumps are broken down. Generally, the process is run until it reaches 98 degrees F., which at this point I found that the large clumps were broken down. Although the process can be run past the point of 98 degrees, it is preferable to keep the temperature at or below this point since at 102 degrees F. the naturally occurring enzymes could be destroyed. Since we have not had the capability of measuring the significance of the effect of these inherent ingredients on metal detox, I thought it best to preserve their integrity with cold process processing at every stage. If necessary, a cooling system could be employed to maintain the temperature in the desired range.

Then 25% of the volume of 42% grain neutral spirits are added to the solution to lower the surface tension and prevent freezing. Although this amount of alcohol can be varied as needed or desired, I stayed with the century old tradition of using 40-42% alcohol for tincturing for human consumption. To make a change in this ratio would involve retesting the effect of the final product so other concentrations have not been tested. Changing the ratio is the same concern as changing the %.

The solution is sonicated using an ultrasonic dismembrator system as described above, so that the temperature of the Chlorella is maintained below zero. The dismembration process is activated and allowed to run for about 20 hours. Quality control may be maintained with a darkfield microscope at 400×. The percentage of cell wall decimation is readily apparent through the microscope. End of processing is determined when about 40% of the clusters and diatoms have been reduced to particulates having a diameter of between about 2 microns and 0.1 microns or less.

The process is stopped at this point because the ultra small particles will cross into the blood via sublingual absorption or across the blood gut barrier especially when in a low surface tension carrier such as alcohol and we want some of the chlorella cell wall to make it through the gastro intestinal tract to absorb toxins there, therefore, some of the diatoms (60% of the yield) are left in tact as they are too large to cross into the blood. Adjusting the ratio of ultra small particles to whole diatoms has been investigated in private in my clinic with the conclusion that a product with exclusively ultra small product goes to the blood and organs first, as noticed by the quick abatement of neurological symptoms, whereas when the concentration favors the whole diatom size relief of gastro intestinal symptoms is experienced first—in heavy metal toxic persons.

The solution is then removed from the process and mixed with 3 ½ parts pure water, 1 part tincture of cilantro and 3 parts 42% grain neutral spirits as a preservative, allowing for a final 20% alcohol content. Alcohol is chosen as the preservative because it further lowers the surface tension of the final solution allowing faster assimilation and better bioavailability. The final mixture is allowed to sit for 2 weeks, which is the standard length of time and percentage of alcohol, to further make a medicinal United States Pharmocopia (USP) tincture out of the solution. Tincturing at this percentage of alcohol to water is known to further release the alkaloid and polysaccharide ingredients of plants and herbs into solution.

The solution is then treated magnetically by exposure to high powered magnets and (the carefree system) to add electrons (negative charge) to the solution and put the tiniest of the particles into colloidal suspension. The solution may then be sterilized with UV light or any other suitable means.

Additional components may be added to the resultant liquid, including mycelials and herbs, sea salt and beneficial human intestinal flora (Polyflor). The mixture may then be allowed to ferment in a sterile environment. Fermentation is typically complete after about 18 hours, or when the pH of the mixture reaches a pH of 3.5.

This mixture may then be mixed with grain neutral spirits (40% alcohol). This mixture can be used by itself or can be combined in any suitable ratio with the previously described mixture.

The Chlorella solution produced by the disclosed methods herein, having either single diatoms homogenously dispersed and/or diatom fragments, permit a person to consume the equivalent surface area of Chlorella cell wall compared to 50 times that amount of commercially available ‘cell wall broken’ chlorella (i.e. chlorella clusters). For example, a therapeutic dose of 2 mls of the chlorella of the present invention twice a day would require a comparable dose of 5,200 mgs of chlorella, which is assuming a person could digest it thoroughly, which they cannot. Taking enzymes such as cellulase and protease might seem to be a solution to this problem. However, such enzymes cleave the fragile and valuable peptydoglycans and polysaccharides, rendering them useless for our purposes of heavy metal and chemical detoxification.

The physical and/or mechanical disruption of Chlorella clusters and diatoms by methods disclosed herein does not. Furthermore, probiotic predigestion with human intestinal flora prepares the ingredients for successful assimilation by a human, given these micro organisms are a part of our digestive process. Concentrated enzymes break the chlorella down to smaller, or ‘too small’ components, i.e. individual amino acids which have little to no effect in heavy metal detoxification, and the value of the complex molecules is lost. If enzymes were the key to this lock, chlorella would have previously proven itself useful for the purpose of heavy metal detoxification as these enzymes pre exist in our GI tracks. Human intestinal flora, on the other hand, is compromised in most persons, and this imbalance accounts for a huge percentage of problems associated with indigestion.

Merely extracting the inherent and already existing cell wall fragments in conventional chlorella requires 500 mg to bind the methyl mercury in the oral cavity compared to 10 mg of chlorella processed according to the present invention. It should be understood that chlorella processed according to the disclosed methods may also be used for eliminating other metals and toxins, such as dioxin and organochlorides.

The present invention has unexpectedly solved a problem facing the industry. All known heavy metal chelators (chemical drugs) mobilize these poisons via the bowel or a combination of the bowel and kidney. They cause a number of problems and undesirable side effects. Primary of which is that if the metals are mobilized via the bowel, there is a risk of resorption and the methylation of mercury (or other toxins) by the intestinal flora. The decimated chlorella of the present invention when used in a solution as described above, are natural, oral, organic solutions to the heavy metal and organochloride toxin problems and mobilize via the urine, and actually improve kidney function in the process, thus by-passing this problem.

The chlorella produced using the process of the present invention can be used in nutritional, pharmaceutical, and cosmetic compositions. Such nutritional and pharmaceutical compositions containing the novel decimated chlorella of the present invention may be formulated and administered in any form suitable for oral, buccal, parental, or enteral administration, such as oral administration or tube feeding.

The formulations are conveniently administered in the form of an alcohol based or an aqueous liquid. The formulations suitable for enteral application are accordingly preferably in aqueous form or in powder or granulate form, including tablet form. The powder or granulate may be conveniently added to water prior to use. In liquid form, the compositions have a solid content of typically from 0.1% to 50% by weight, preferably from 1% to 10% by weight.

As a drink, the compositions may be obtained by any manner known, e.g. by admixing the Chlorella extract with an energy source such as carbohydrates, fats and nitrogen sources. The nutritional compositions may be in the form of a complete formula diet (in liquid or powder form), such that when used as sole nutrition source, essentially all daily caloric, nitrogen, fatty acids, vitamin, mineral and trace element requirements are met. However, the nutritional compositions of the invention are preferably intended for use as a dietary supplement.

Pharmaceutical compositions of the invention may also be formulated in a single-dose format, where they comprise chlorella extracts and a pharmaceutically acceptable carrier. Such pharmaceutical compositions are suitable for enteral administration, such as oral, nasal or rectal administration. Suitable compositions may be in liquid form or solid form. Dosage of liquid compositions are typically from 0.1% to 50% by weight, preferably from 1% to 10% by weight of chlorella extract. Dosage of solid compositions are typically from 0.2 mg/kg to 200 mg/kg, preferably from 1 mg/kg to 10 mg/kg of chlorella extract The compositions may be in the form of tablets, hard and soft capsules, and sachets.

Suitable carriers are known in the art. They comprise fillers such as sugars or cellulose, binders such as starch, and disintegrators if required.

Such cosmetic compositions containing the novel decimated chlorella of the present invention may be formulated and administered in any form suitable including shampoo, conditioner, facial masque, creams, lotions, sprays, toothpaste, mouthwash and any other form where a cleansing, protective and nourishing effect are desired. It was further found that chlorella in this novel form behaves as a sun block, effectively blocking both UVA and UVB solar radiation.

It will be apparent to one of ordinary skill in the art that various modifications and variations can be made in configuration or formulation of the present invention without departing from the scope or spirit of the invention. Thus, it is intended that the present invention cover all such modifications and variations, provided they come within the scope of the following claims and their equivalents.

I claim as follows:

1. A solution comprising water, alcohol and homogenously dispersed Chlorella diatoms having a diameter of less than about 5 microns.

2. The solution of claim 1, further comprising Chlorella cell wall fragments having a diameter of less than about 2 microns.

3. The solution of claim 2, wherein at least a portion of said fragments have a diameter of less than about 0.5 microns.

4. The solution of claim 3, wherein a majority of said fragments have a diameter of less than about 0.1 microns.

5. The solution of claim 1, wherein said solution includes at least about 20% by volume alcohol.

6. The solution of claim 5, further comprising at least one of a mycelia and an herb.

7. The solution of claim 5, further comprising sea salt.

8. The solution of claim 5, further comprising at least about 10% by volume Cilantro.

9. The solution of claim 2, further comprising cell wall broken probiotics having a diameter of less than about 5 microns.

10. The solution of claim 2, wherein said solution includes between about 0.1% by weight and about 10% by weight of said diatoms and said fragments.

11. Nanonized cell wall chlorella powder comprising cell wall fragments having a diameter of less than about 2 microns.

12. The powder of claim 11, wherein at least a portion of said cell wall fragments have a diameter of less than about 0.1 microns.

13. A method of pulverizing Chlorella, comprising the steps of:
providing a liquid solution comprising alcohol and chlorella;
maintaining the solution at a temperature of less than about zero degrees Celsius while sonicating the solution using an ultrasonic dismembrator for a sufficient period to achieve a solution with homogenously dispersed chlorella diatoms having a diameter of less than about 5 microns.

14. The method of claim 13, including the step of super cooling the solution to about −15° C. prior to said maintaining and sonicating step.

15. The method of claim 13, including the step of maintaining the Chlorella in the solution at a temperature of about −1° C. or colder during said maintaining and sonicating step.

16. The method of claim 15, including the further step of terminating sonication prior to fragmenting a majority of the chlorella diatoms into colloids having a diameter of less than about 2 microns.

17. The method of claim 15, including the further step of terminating sonication after fragmenting a majority of the chlorella diatoms into colloids having a diameter of less than about 2 microns.

18. The method of claim 17, including the further step of fermenting the sonicated solution using probiotic predigestion.

19. The method of claim 18, wherein human beneficial intestinal flora are used during said fermenting step.

20. The method of claim 18, wherein yeast is used during said fermenting step.

21. The method of claim 18, including the step of terminating fermentation after the solution reaches a pH of about 3.5 or higher.

22. The method of claim 1, including the further step of filtering the sonicated solution to remove colloids in the solution having a diameter of greater than about 0.1 microns.

23. The method of claim 18, including the further step of filtering the sonicated solution to remove colloids in the solution having a diameter of greater than about 0.1 microns.

24. The method of claim 18, including the further step of grinding the sonicated solution and thereby disperse the colloids in the solution.

25. The method of claim 13, including the further step of adding at Cilantro to the solution.

26. The method of claim 13, including the further step of adding probiotics to the solution.

27. The method of claim 13, including the further step of exposing the sonicated solution to high powered magnets to add electrons to the solution.

28. The method of claim 13, including the further step of exposing the sonicated solution to an electrical ionizer.

29. The method of claim 13, including the further step of exposing the sonicated solution using UV light.

30. The method of claim 13, including the step of providing a solution comprising at least about 80% by weight of alcohol.

31. A method of removing heavy metals from a patient, comprising the step of orally administering a liquid solution to the patient, the solution comprising chlorella fragments having a diameter of less than about 5 microns, the fragments binding to a heavy metal in the patient and thereafter being excreted via the patient's urinary tract.

32. The method of claim 31, wherein the solution further comprises cell wall broken probiotics and tincture of cilantro during said administering step.
